(19) United States
(12) Patent Application Publication
(10) Pub. No.: US 2026/0078433 A1
(43) Pub. Date: Mar. 19, 2026

(54) USE OF RNASEHII AND PROBES FOR SENSITIVE AND SPECIFIC DETECTION OF SINGLE NUCLEOTIDE POLYMORPHISMS

(71) Applicant: GoDx, Inc., Madison, WI (US)

(72) Inventors: Xiao JIANG, Madison, WI (US); Chang Hee KIM, Waunakee, WI (US)

(21) Appl. No.: 19/328,896

(22) Filed: Sep. 15, 2025

Related U.S. Application Data
(60) Provisional application No. 63/694,643, filed on Sep. 13, 2024.

(51) Int. Cl. C12Q 1/6827; C12Q 1/6804; C12Q 1/6823; C12Q 1/6844; C12Q 1/689

(52) U.S. Cl. CPC C12Q 1/6827; C12Q 1/6804; C12Q 1/6823; C12Q 1/6844; C12Q 1/689

(57) ABSTRACT

The present invention provides compositions, probes, methods, and point-of-need testing devices for detecting a single nucleotide polymorphism in target DNA sequence. The compositions comprise a probe, a forward primer, a reverse primer, a RNascHII, and a recombinase polymerase amplification reagent mix.

### CROSS-REFERENCE TO RELATED APPLICATIONS

This application claims priority to U.S. Provisional Application No. 63/694,643, filed on Sep. 13, 2024, the contents of which are incorporated by reference herein in their entirety.

### STATEMENT REGARDING FEDERALLY SPONSORED RESEARCH

This invention was made with government support under grant number R43 AI157577 awarded by the National Institutes of Health. The government has certain rights in this invention.

### REFERENCE TO AN ELECTRONIC SEQUENCE LISTING

The contents of the electronic sequence listing (165369_00042.xml; Size: 35,880 bytes; and Date of Creation: Sep. 8, 2025) are herein incorporated by reference in its entirety.

### BACKGROUND

Single-nucleotide polymorphisms (SNPs) are powerful tools that can be used for a variety of genetic and bioinformation purposes. For example, SNPs can provide insight into the prognosis, progression, and/or treatment response of diseases, and can enable the identification specific pathogen strains and mutations associated with antimicrobial resistance (AMR). RNase H-dependent PCR (rhPCR) is one method that enables SNP detection.³⁻⁵ However, there are disadvantages associated with PCR-based methods, including long run times and the requirement of expensive laboratory equipment (e.g., thermocyclers with fluorescent detection capability).

Recombinase polymerase amplification (RPA) is an isothermal amplification technique that is performed under a single incubation temperature (37-40° C.) and has the capability of amplifying as low as 1-10 DNA copies in less than 20 min.¹ Because amplification is performed at a single temperature, RPA eliminates the need for a thermocycler.

In RPA, recombinase-primer complexes are formed by primers and recombinase proteins such as the T4 bacteriophage UvsX. The recombinase-primer complexes scan the target duplex DNA for homology, and strand exchange between the duplex and the primer forms at the cognate site with the assistance of a single-stranded binding protein, such as gp32, and a recombination mediator protein, such as the T4 bacteriophage UvsY. The gp32 stabilizes the displaced template strand, thus keeping the primer from ejection through branch migration. The recombinase disassembly leaves the 3-prime (3′) end of the annealed primer open for a strand displacing DNA polymerase such as the large fragment of Bacillus subtilis polymerase I (Bsu). The polymerase then elongates the primer to generate amplicons.

One method of using RPA involves using an endonuclease IV (nfo) and a probe with 5-prime (5′) end tag, an abasic site in the middle, and a 3-prime end blocker to increase the specificity for LFA RPA detection.¹ However, this approach cannot meet the specificity required for SNP genotyping. Another method uses one or both primers containing a RNA base and a 5-prime blocker to eliminate dimer formation in the RPA reaction.² However, this approach has not been used to detect SNPs. Thus, while RPA offers certain improvements over PCR-based approaches (lower costs, faster run times), no RPA methods have been developed that exhibit the high specificity required to detect SNPs.

Thus, there remains a need for improved compositions and methods that can specifically detect SNPs and can be employed in resource-limited settings.

### SUMMARY

In a first aspect, the present invention provides compositions for detecting a single nucleotide polymorphism (SNP) in a target DNA sequence. The compositions comprise (a) a probe comprising a polynucleotide complementary to the target DNA sequence, the polynucleotide comprising, in order from 5′ to 3′: a first DNA sequence, a RNA base complementary to the SNP, a second DNA sequence, and optionally a mismatched DNA base, wherein the polynucleotide is conjugated to a 5′ tag and a 3′ blocker, (b) a forward primer and a reverse primer; (c) a RNaseHII; and (d) a recombinase polymerase amplification (RPA) reagent mix.

In a second aspect, the present invention provides methods for detecting a single nucleotide polymorphism (SNP) in a target DNA sequence. The methods comprise (a) obtaining a sample suspected of comprising the target DNA sequence; (b) contacting the target DNA sequence with a composition of the present invention; (c) amplifying the target DNA sequence, wherein the amplifying is performed isothermally, and (d) detecting an amplicon of the target DNA sequence.

In a third aspect, the present invention provides probes for detecting a single nucleotide polymorphism (SNP) in a target DNA sequence. The probes comprise a polynucleotide complementary to the target DNA sequence, the polynucleotide comprising in order from 5′ to 3′: (a) a first DNA sequence of 10 to 40 bases; (b) a RNA base complementary to the SNP; (c) a second DNA sequence of 4 to 10 bases; and (d) optionally, a mismatched DNA base, wherein the polynucleotide is conjugated at the 5′ end to a tag and at the 3′ end to a blocker.

### BRIEF DESCRIPTION OF THE DRAWINGS

FIG. 1 is a schematic depiction of the mechanism of the RNaseHII assay, using LFA detection.

FIGS. 2A-2B demonstrate that the designed primers and probes enable detection of SNPs in Neisseria gonorrhoeae using RPA and RNaseHII. FIG. 2A depicts the results from three LFAs and demonstrates the detection of three SNP mutations associated with AMR in Neisseria gonorrhoeae (F505L, A511V, and A517G SNP mutations in the penA gene). FIG. 2B depicts gel electrophoresis images confirming that, following the RNaseHII/RPA assay, the primer amplicons (top square) are present in both (1) samples containing the nucleotide present in the wild-type gene sequence and (2) samples containing the AMR target mutation, while the probe amplicons (bottom square) are present only in the samples containing the AMR target mutation.

FIGS. 3A-3B demonstrate that the designed primers and probes enable detection of SNPs in Salmonella enterica using RPA and RNaseHII. FIG. 3A depicts the results from two LFAs and demonstrates the detection of two SNP mutations associated with AMR in Salmonella enterica (R717L and R717Q SNP mutations in the acrB gene). FIG. 3B depicts gel electrophoresis images confirming that, following the RNascHII/RPA assay, the primer amplicons (top square) are present in samples containing the AMR target mutation and samples that do not contain the AMR target mutation, while the probe amplicons (bottom square) are present only in the samples containing the AMR target mutation.

FIGS. 4A-4B demonstrate that the designed primers and probes enable detection of SNPs in Campylobacter jejuni using RPA and RNascHII. FIG. 4A depicts the results from a LFA and demonstrates the detection of T861 (C257T) SNP mutation associated with AMR in the gyrA gene of Campylobacter jejuni. FIG. 4B depicts gel electrophoresis images confirming that, following the RNascHII/RPA assay, the primer amplicons (top square) are present in both (1) samples containing the nucleotide present in the wild-type gene sequence and (2) samples containing the AMR target mutation, while the probe amplicons (bottom square) are present only in the samples containing the AMR target mutation.

FIGS. 5A-5B further demonstrate SNP detection in Campylobacter jejuni using the designed primers and probes, RPA, and RNascHII. FIG. 5A depicts the results from a LFA and demonstrates the detection of an additional SNP mutation associated with AMR in Campylobacter jejuni (A2075G SNP mutation in the 23S rRNA gene). FIG. 5B depicts gel electrophoresis images confirming that, following the RNaseHII/RPA assay, the primer amplicons (top square square) are present in both (1) samples containing the nucleotide present in the wild-type gene sequence and (2) samples containing the AMR target mutation, while the probe amplicons (bottom square) are present only in the samples containing the AMR target mutation.

FIG. 6 depicts LFA results demonstrating that the T86I (C257T) SNP mutation in gyrA can be detected using the designed primers and probes, RPA, and Escherichia coli RNascHII. E. coli RNaseHII preferentially cleaved the probe at the complementary RNA base, producing a stronger test line signal in the mutant samples than in the wild-type samples, even when a high copy number (2×10⁴ CFU) of wild-type sample was presented.

### DETAILED DESCRIPTION

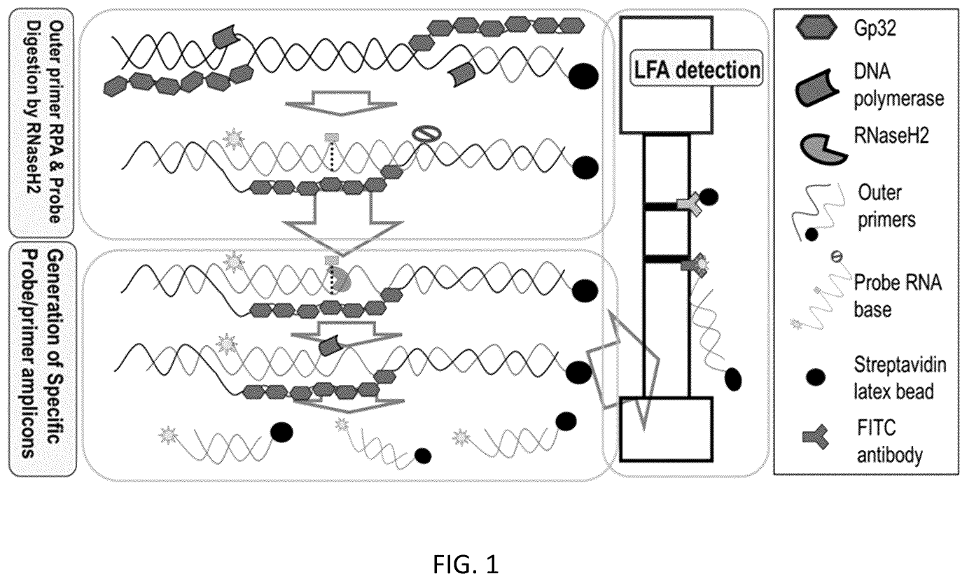

Disclosed herein are compositions, probes, and methods for detecting a single nucleotide polymorphism (SNP) in a target DNA sequence, utilizing recombinase polymerase amplification (RPA) in the presence of RNaseHII (illustrated in FIG. 1). As used herein, “single nucleotide polymorphism” refers to a genetic variant resulting from the substitution of a single nucleotide for a different nucleotide. The compositions, probes, and methods can be used to perform rapid detection of SNPs without the need for expensive laboratory equipment or instruments, or specially trained laboratory technicians. The compositions, probes, and methods may suitably be used to detect the presence of a target nucleic acid or determine if a patient is infected with a pathogen, including a pathogen associated with antimicrobial resistance. In addition, the compositions, probes, and methods may be utilized in a point-of-need (PON) testing device.

### Compositions and Probes

The present invention provides compositions for detecting a single nucleotide polymorphism (SNP) in a target DNA sequence. The compositions comprise a probe, a forward primer, a reverse primer, a RNaseHII, and a recombinase polymerase amplification (RPA) reagent mix. The compositions may be utilized in a RNaseHII/RPA assay, as further described in the methods section below.

A “target DNA sequence” is a DNA sequence containing a SNP that is indicative of an origin or source. The target DNA sequence amplified using the compositions and methods of the present invention may be derived from genomic DNA (e.g., DNA encoding a protein, open reading frames, or regulatory sequences), mitochondrial DNA, extracellular DNA, plasmid DNA, or cell-free fetal DNA. In some embodiments, the target DNA sequence is indicative of the presence of a particular organism, such as a pathogen (e.g., a bacterium, a fungus, a virus, or a protist). As used herein, a “pathogen” is any microorganism capable of causing disease in a patient or subject. Any pathogen may be detected using the disclosed methods, including, for example, a bacterium, a fungus, a virus, or a protist. Any pathogen may be detected using the present invention, including, without limitation, Escherichia, Campylobacter, Clostridium difficile, Enterotoxigenic E. coli (ETEC), Enteroaggregative Escherichia coli (EAggEC), Shiga-like Toxin producing E. coli (STEC), Salmonella, Shigella, Vibrio, Yersinia enterocolitica, Adenovirus, Norovirus, Rotavirus A, Cryptosporidium parvum, Entamoeba histolytica, Giardia lamblia, Clostridia, Staphylococcus aureus, Klebsiella pneumonia, influenza, Zika, dengue, chikungunya, West Nile virus, Japanese encephalitis, malaria, HIV, HINI, HPV, Hepatitis, Ebola, Streptococcus, Neisseria, and Mycobacterium.

In some embodiments, the target DNA sequence contains a SNP that is associated with antimicrobial resistance (AMR). To this end, the primers and probes used in present the invention are designed to differentiate between resistant alleles and wild-type alleles that suggest whether the pathogen is resistant or susceptible to a particular drug. Antibiotic susceptibility and resistance will be defined according to CDC and Clinical Laboratory Standards Institute Guidelines. If a strain detected in a patient is found to be resistant to a certain antimicrobial drug, alternative antimicrobials could be prescribed. Such results enable antibiotic stewardship and effective treatment. A skilled artisan will readily appreciate SNPs that are associated with AMR and can be detected using the present invention, including, without limitation, SNP mutations in the penA gene of Neisseria gonorrhoeae, the acrB gene of Salmonella enterica, the gyrA gene of Campylobacter jejuni, and the 23S rRNA gene of Campylobacter coli. In some embodiments, the target DNA sequence is a penA gene of Neisseria gonorrhoeae containing a F505L, A511V, and/or A517G SNP mutation. In some embodiments, the target DNA sequence is an acrB gene of Salmonella enterica containing a R717L or R717Q SNP mutation. In some embodiments, the target DNA sequence is a gyrA gene of Campylobacter jejuni containing a T861 SNP mutation. In some embodiments, the target DNA sequence is a 23S rRNA gene of Campylobacter coli containing a A2075G SNP mutation. In some embodiments, the target DNA sequence comprises the sequence set forth in any one of SEQ ID NOs: 6-7, 12-14, 18-19, and 23-24, or comprises a sequence having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 6-7, 12-14, 18-19, and 23-24.

In other embodiments, the target DNA sequence is indicative of the presence or absence of a disease or condition. In yet other embodiments, the target DNA sequence is indicative of the prognosis, progression, or response to treatment for a disease or condition. As used herein, “indicative” or “indicates” means to point to or be a sign of an origin or source, whether alone or in combination with additional target sequences or other information. A skilled artisan will readily appreciate SNPs that are associated with disease incidence, prognosis, progression, or response to treatment that can be detected using the present invention. The target DNA sequence may be found anywhere in the genome of a specific organism, but it should be specific to said organism.

The probe comprises a polynucleotide complementary to the target DNA sequence. A polynucleotide is “complementary” to the target DNA sequence if it has the ability to hybridize with the target DNA sequence via the formation of hydrogen bonds between specific nucleotides (i.e., A binds to T or U and G binds to C), forming a double-stranded molecule. A nucleotide that does not hybridize with the target DNA sequence in this manner is referred to as a “mismatch.” In some embodiments, each nucleotide of the polynucleotide is complementary to the target DNA sequence. In other embodiments, at least 93% of the polynucleotide (e.g., 14 of 15 nucleotides) is complementary to the target DNA sequence. The polynucleotide comprises, in order from 5′-3′, a first DNA sequence, a RNA base complementary to the SNP, a second DNA sequence, and optionally a mismatched DNA base. An exemplary probe is shown below. “D” denotes a complementary DNA base (the first DNA sequence is italicized; the second DNA sequence is in bold); M denotes the optional mismatched DNA base, and “r” denotes the RNA base complementary to the SNP:
(5′) Tag-DDDDDDDDDDDDDDDDDDDDDDDDDDDDDDrDDDDM-Blocker (3′)

The first DNA sequence and the second DNA sequence each comprises or consists of DNA bases complementary to the target DNA sequence. The first DNA sequence may have a length of 1, 5, 10, 15, 20, 25, 30, 35, 40, 45, 50, 55, or 60 bases, or a length within a range bounded by any of the foregoing. In some embodiments, the first DNA sequence has a length of 10 to 40 bases. In some embodiments, the first DNA sequence has a length of 28 to 35 bases. The second DNA sequence may have a length of 1, 2, 3, 4, 5, 6, 7, 8, 9, 10, 11, 12, 13, 14, or 15 bases, or a length within a range bounded by any of the foregoing. In some embodiments, the second DNA sequence has a length of 4-10 bases. In some embodiments, the second DNA sequence has a length of 4-6 bases. In some embodiments, the second DNA sequence is flanked at the 5′ end by the mismatched DNA base. The RNA base (rA, rU, rC, or rG) is located between the first DNA sequence and the second DNA sequence and is complementary to the SNP. In embodiments, the RNA base—when hybridized to the SNP—serves as a RNaseHII cleavage site, enabling the RNaseHII to selectively cleave the probe at the 5′ side of the RNA base (i.e., between the RNA base and the first DNA sequence). Thus, the probe is configured to be selectively cleaved by a RNaseHII.

The polynucleotide is conjugated to a 5′ tag and a 3′ blocker. As used herein, a “5′ tag” refers to a detectable labeling moiety that is conjugated to the 5′ end of the polynucleotide. Suitable labeling moieties are known in the art and include, without limitation, a gold nanoparticle, a protein binding ligand, a hapten, an antigen, a fluorescent compound, a dye, a radioactive isotope, and an enzyme. The 5′ tag may be detectable by any detection apparatus or method that provides a readout that indicates whether a target DNA sequence is present in a sample. In some embodiments, the 5′ tag is detectable by electrophoresis and/or LFA. In some embodiments, the tag is a fluorescein isothiocyanate (FITC) tag. As used herein, a “3′ blocker” refers to a moiety that is conjugated to the 3′ end of the probe and that prevents extension of the probe by DNA polymerase. The 3′ blocker may additionally render the probe resistant to exonuclease digestion. Suitable 3′ blockers include, without limitation, phosphorothioate bonds, 3′-inverted thymidine (dT), 5′-inverted dT, and sugar moieties (e.g., ribo, 2′-methoxy, and 2′-methoxyethyl groups). In some embodiments, the 3′ blocker is a 3′ inverted dT blocker.

In some embodiments, the polynucleotide comprises the sequence set forth in of any one of SEQ ID NOs: 3-5, 10-11, 17, and 22, or comprises a sequence having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 3-5, 10-11, 17, and 22.

The probe may be present in the compositions at a concentration of about 25 nM, about 50 nM, about 100 nM, about 150 nM, about 200 nM, about 250 nM, about 300 nM, about 350 nM, about 400 nM, about 450 nM, about 500 nM, or about 550 nM, or at a concentration within a range bounded by any of the foregoing. In some embodiments, the probe is present at a concentration of about 50 nM to about 500 nM. In some embodiments, the probe is present at a concentration of about 250 nM.

The compositions of the present invention further comprise a forward primer and a reverse primer. As used herein, a “primer” is a nucleic acid designed to hybridize to the end of the target DNA sequence, which is extended by DNA polymerases during amplification. A “forward primer” hybridizes to the antisense strand of double-stranded DNA, which runs in the 3′ to 5′ direction, whereas a “reverse primer” hybridizes to the sense strand of double-stranded DNA, which runs in the 5′ to 3′ direction. Methods for designing primers are known in the art. Many resources, including, without limitation, literature publications and NCBI databases such as “BLAST”, may be used to guide primer design. The binding site for each primer should be unique to the target DNA sequence and have minimal homology to other sequences to ensure specific amplification of the target DNA sequence. In some embodiments, the cleaved probe serves as an inner forward primer in the RPA reaction.

In some embodiments, the reverse primer comprises a primer tag that is conjugated to the 5′ end of the primer. In some embodiments, the primer tag is a biotin tag. The primer tag may facilitate capture of the primer by a capture agent, including, without limitation, by affinity binding. For instance, when the compositions are used in conjunction with LFA, the biotin-tagged reverse primers may be captured by LFA strips comprising streptavidin-gold nanoparticles (AuNPs). In some embodiments, the forward primer and reverse primer are designed to hybridize to the target DNA sequence set forth in any one of SEQ ID NOs: 6-7, 12-14, 18-19, and 23-24, or to a target DNA sequence having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 6-7, 12-14, 18-19, and 23-24. In some embodiments, the forward primer comprises the sequence set forth in any one of SEQ ID NOs: 1, 8, 15, and 20, or comprises a sequence having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 1, 8, 15, and 20. In some embodiments, the reverse primer comprises the sequence set forth in any one of SEQ ID NOs: 2, 9, 16, and 21, or comprises a sequence having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 2, 9, 16, and 20.

The forward primer and the reverse primer may be present in the compositions at a concentration of about 50 nM, about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1000 nM, or about 1050 nM, or at a concentration within a range bounded by any of the foregoing. The forward primer and the reverse primer may be present in the composition at the same concentration, or at different concentrations. In some embodiments, the forward primer and the reverse primer are present in the composition at a concentration of about 100 nM to about 1000 nM. In some embodiments, the forward primer and the reverse primer are present in the composition at a concentration of about 500 nM.

The compositions of the present invention comprise a RNaseHII. As used herein, “RNaseHII” or “Ribonuclease HII” is an endonuclease enzyme belonging to the Ribonuclease H family, which is capable of selectively or preferentially cleaving a single RNA base—or a strand of RNA bases—present in a double-stranded DNA. RNaseHII preferentially hydrolyzes DNA-RNA phosphodiester bonds located 5′ to the RNA, resulting in a terminal DNA base having a 3′ hydroxyl moiety and a terminal RNA base having a 5′ phosphate moiety. In some embodiments, the RNaseHII is thermostable and is capable of selectively cleaving the probe at a temperature of 37 to 40° C. The RNaseHII used in the compositions can be from any source. In some embodiments, the RNaseHII is derived from Pyrococcus abyssi, Thermus thermophilus, Chlamydia pneumoniae, or Escherichia coli. In some embodiments, the RNaseHII is an enzyme of any one of SEQ ID NOs: 25-28, or is an enzyme having at least 70%, at least 75%, at least 80%, at least 85%, at least 88%, at least 90%, at least 92%, at least 95%, at least 96%, at least 97%, at least 98%, or at least 99% sequence identity to any one of one of SEQ ID NOs: 25-28.

The RNaseHII may be present in the compositions at any suitable concentration. In some embodiments, the RNaseHII is present in the compositions at a concentration of about 0.1 mU/μl, about 1 mU/μl, about 5 mU/μl, about 10 mU/μl, about 15 mU/μl, about 20 mU/μl, about 25 mU/μl, about 30 mU/μl, about 35 mU/μl, about 40 mU/μl, about 45 mU/μl, about 50 mU/μl, about 75 mU/μl, about 100 mU/μl, about 125 mU/μl, about 150 mU/μl, about 175 mU/μl, about 200 mU/μl, about 225 mU/μl, about 250 mU/μl, about 275 mU/μl, or at a concentration within a range bounded by any of the foregoing. In some embodiments, the RNaseHII is present in the composition at a concentration of about 6.6 mU/μl, about 26.4 mU/μl, or about 125 mU/μl.

The compositions comprise a recombinase polymerase amplification (RPA) reagent mix. As used herein, the phrase “recombinase polymerase amplification reagent mix” refers to a mixture comprising deoxynucleotide triphosphates (dNTPs) and additional reagents (beyond the forward primer and reverse primer) needed to perform the RPA reaction. In some embodiments, the RPA reagent mix comprises a recombinase protein, a single-stranded binding protein, a recombination mediator protein, and/or a strand-displacing polymerase.

A “recombinase protein” is an enzyme that catalyzes DNA exchange reactions. In RPA, rather than melting the dsDNA in a sample to make primer binding sites accessible as you would in PCR, a recombinase is used to load the primers. The recombinase forms a complex with a primer and scans the dsDNA for a homologous sequence. When the homologous sequence is found, the recombinase performs DNA strand exchange, displacing the complementary strand and allowing the primer to hybridize to the homologous sequence. The composition may comprise any suitable recombinase protein. In some embodiments, the recombinase is UvsX from T4 bacteriophage. In other embodiments, the recombinase is RecA, from E. coli or RecT from E. coli or a homolog thereof.

A “single-stranded DNA binding protein (SSB)” is a protein that binds to and stabilizes single-stranded DNA. In the compositions, the SSB can be used to stabilize the displaced complementary strand so that it does not displace the bound primer. The composition may comprise any suitable SSB. In some embodiments, the SSB is gp32 from T4 bacteriophage.

A “recombination mediator protein” is a protein that regulates homologous recombination (i.e., the exchange of strands between homologous DNA molecules). The composition may comprise any suitable recombination mediator protein. In some embodiments, the recombination mediator protein is UvsY from T4 bacteriophage. UvsY stimulates the activity of the recombinase UvsX, lowers the critical concentration of UvsX that is required for activity, and promotes strand exchange. In other embodiments, the recombination mediator protein is RecO and/or RecR from E. coli, which stabilize the binding of RecA to single-stranded primers.

A “strand-displacing polymerase” is an enzyme that catalyzes the formation of DNA, with the ability to displace downstream DNA encountered during synthesis. The composition may comprise any suitable strand-displacing polymerase, including, without limitation, phi29, Bst, Bsm, Bsu, and Klenow fragment. In some embodiments, the stand-displacing polymerase is the large fragment of Bacillus subtilis polymerase I (Bsu).

In the Examples, the inventors utilized a commercial RPA kit (i.e., the TwistAmp® Liquid Basic Kit from TwistDx) that includes the necessary recombinase, recombination mediator protein, SSB, and stand-displacing DNA polymerase to perform RPA. Thus, in some embodiments, the RPA reagent mix is provided in the form of a commercial kit. The RPA reagent mix may further comprise additional components, including cofactors, buffering agents, crowding agents, amplification enhancers, or any combination thereof. As used herein, a “cofactor” is a substance other than the substrate that is essential for the activity of an enzyme. Exemplary cofactors include magnesium, which functions as a cofactor for a variety of polymerases. The cofactor may be introduced to the amplification reaction as a salt, e.g., MgSO₄ or MgCl₂. As used herein, a “buffering agent” comprises a weak acid or base and is used to maintain the acidity (pH) of a solution near a chosen value after the addition of another acid or base. Suitably, the buffering agent may be selected from Tris-HCl, (NH₄)₂SO₄, or KCl. As used herein, an “amplification enhancer” is a substance that enhances amplification specificity, efficiency, consistency, and/or yield. Exemplary amplification enhancers include dimethyl sulfoxide, glycerol, formamide, polyethylene glycol, N,N,N-trimethylglycine (betaine), bovine serum albumin, tetramethylammonium chloride, a detergent, or combinations thereof. Suitably, the detergent is a nonionic detergent such as Tween 20 or Triton X-100. In some embodiments, the RPA reagent mix comprises Triton X-100. The Triton X-100 may be present in the compositions at a concentration of about 0.0007%, about 0.0008%, about 0.0009%, about 0.001%, about 0.002%, about 0.003%, about 0.004%, about 0.005%, about 0.006%, about 0.009%, about 0.008%, about 0.009%, about 0.01%, or about 0.02%, or at a concentration within a range bounded by any of the foregoing. In some embodiments, the Triton X-100 is present in the compositions at a concentration of about 0.00075 to about 0.01%.

### Methods

Also provided herein are methods for detecting a single nucleotide polymorphism (SNP) in a target DNA sequence, using the compositions and probes disclosed herein. As illustrated in FIG. 1, the RPA reaction proceeds from the forward and reverse primers, producing an amplicon. The probe then invades and hybridizes to a position downstream of the forward primer. If the RNA base is a match with the targeted SNP site at the amplicon, the RNaseHII cleaves the probe at the 5′ side of RNA base, releasing the 3′ blocker of the probe. Release of the blocker then allows polymerase to proceed, producing a detectable amplicon from the probe and reverse primer, with the cleaved probe functioning as an inner forward primer. The amplicon may be detected via the 5′ tag on the probe.

The methods comprise obtaining a sample suspected of comprising the target DNA sequence. As used herein, a “sample” is a substance that comprises or may comprise nucleic acids. The samples used with the present invention may be liquid, solid, or semi-solid. In some embodiments, the sample comprises cells in culture. In other embodiments, the sample is a biological sample obtained from a subject, e.g., a patient. Exemplary patient samples include stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool, stool water, pancreatic juice, lavage fluids from sinus cavities, bronchopulmonary aspirates, blastocyl cavity fluid, umbilical cord blood, a skin swab sample, a throat swab sample, a genital swab sample, a rectal swab sample, and an anal swab sample. In some embodiments, the sample is an environmental sample. Exemplary environmental samples include soil, rock, plant, water, and air samples. In other embodiments, the sample is a food sample or another consumable. Exemplary food samples include meat, dairy, and produce samples. In some embodiments, the sample comprises nucleic acids that were extracted or purified from a biological material.

In some embodiments, the sample collection apparatus will utilize a Puritan® HydraFlock swab to increase absorption and retention of cellular material. Since this fully saturated swab can hold approximately 250 μL of sample, the addition of the swab to a lysis buffer standardizes sample input without requiring any measurement or transfer of infectious liquids.

In some embodiments, the methods are performed with minimal processing of the sample. For example, the methods may be performed with no molecular purification or substrate filtering prior to the disclosed RPA/RNaseHII assay. The disclosed methods, compositions, and devices can be used with or without lysing the cells within a sample.

In other embodiments, the sample is processed prior to the RPA/RNaseHII assay. For instance, for some samples (e.g., vaginal or cervical swabs (self-collected or clinician-collected), first-catch urine samples, swabs of penile or vaginal discharge, swabs of external genitalia, throat swabs, swabs of stool samples, or anal swabs), the cellular content may be lysed to allow the nucleic acids to be extracted for efficient amplification of target genes. As used herein, a “lysis buffer” is a composition capable of breaking down or disrupting a cellular membrane. Exemplary lysis buffers may comprise, without limitation, chaotropic salts (e.g., guanidine thiocyanate, alkali metal perchlorates, alkali metal iodides, alkali metal trifluoroacetates, alkali metal trichloroacetates, alkali metal thiocyanates, urea, guanidine HCl, guanidine thiocyanate, guanidium thiosulfate, TrisHCl, magnesium acetate, and thiourea), lytic enzymes (e.g., beta glucurondiase, glucanase, glusulase, lysozyme, lyticase, mannanase, mutanolysin, zymolase, cellulase, lysostaphin, pectolyase, and streptolysin O), and detergents (e.g., 3-[(3-cholamidopropyl)dimethylammonio]-1-propanesulfonate, octyl-β-thioglucopyranoside, octyl-glucopyranoside, 3-(4-heptyl) phenyl 3-hydroxy propyl)dimethylammonio propane sulfonate, 3-[N,N-dimethyl (3-3-myristoylaminopropyl) ammonio] propanesulfonate, (decyldimethylammonio) propanesulfonate inner salt, 3-(dodecyldimethylammonio) propanesulfonate inner salt, 3-(N,N-dimethylmyristylammonio) propanesulfonate, and n-dodecyl a-D-maltoside). In some embodiments, the lysis buffer comprises TrisHCl and magnesium acetate. In some embodiments, the TrisHCl is present in the lysis buffer at a concentration of about 10 mM and/or the magnesium acetate is present in the lysis buffer at a concentration of about 14 mM.

In some embodiments, the sample is collected with a swab and mixed with a binding/washing buffer to elute the sample, producing a sample eluate. In some embodiments, the binding/wash buffer comprises phosphate buffered saline (PBS), bovine serum albumin (BSA), and/or Tween20. In further embodiments, the binding/wash buffer comprises 1×PBS, 1% BSA, 0.05% Tween20, and 0.05% NaN3. The sample eluate may be further processed to purify and/or enrich the target DNA using any suitable purification and/or enrichment methods known in the art. In some embodiments, the sample eluate comprises non-target cells and target cells. As used herein, a “non-target cell” is a cell derived from an organism that is not expected to comprise the target DNA sequence, while a “target cell” is a cell derived from an organism suspected of comprising the target DNA sequence. For example, when the methods are used to detect a SNP in a Salmonella enterica target DNA sequence, a target cell refers to a cell derived from Salmonella enterica, while a non-target cell may refer to a cell derived from an organism other than Salmonella enterica, including, without limitation, cells derived from other pathogens.

In some embodiments, the sample eluate is (1) contacted with a first set of magnetic beads coated with antibodies capable of binding to a non-target cell and (2) incubated on rotation to capture non-target cells present in the eluate, producing a treated eluate. The treated eluate may be transferred to a vessel and (1) contacted with a second set of magnetic beads coated with antibodies capable of binding to a target cell and (2) incubated on rotation, producing bound target cells. In some embodiments, the bound target cells are washed one or more times with the binding/washing buffer, and then incubated in the lysis buffer, producing a lysed sample. In some embodiments, the bound cells are incubated in the lysis buffer for about 15 minutes at a temperature of about 95° C.

In some embodiments, the sample is subjected to a DNA preprocessing step prior to the RPA/RNaseHII assay, for example, shearing, sonication, nebulization, enzymatic digestion, isolation, extraction, enrichment, or purification. The sample may be subjected to any suitable DNA preprocessing methods known in the art.

The methods further comprise contacting the target DNA sequence with a composition of the present invention, to generate an amplification reaction mix, and amplifying the target DNA sequence in the amplification reaction mix. At the time the target DNA sequence is contacted with the composition, the target DNA sequence may present in, without limitation, the unprocessed or minimally processed sample, or an aqueous sample resulting from sample processing (e.g., the lysed sample). The amplifying is performed under conditions suitable for amplification. The term “conditions suitable for amplification” refers to conditions in which incubation of the amplification reaction mix results in the generation of the target amplicon if the target DNA sequence is present in the sample. These conditions include a suitable reaction time and concentrations of the various reagents included in the amplification reaction mix for amplification to occur. The conditions suitable for amplification will depend on the selected amplification method.

In the methods, the amplifying is performed isothermally. As used herein, “isothermally” or “under isothermal conditions” means that reaction is conducted at a relatively constant temperature. Suitably, the reaction is conducted with temperature fluctuations less than #10° C., +5° C., or +2° C. The amplifying may be performed without the use of a thermocycler. In some embodiments, the amplifying is performed without any equipment requiring a power supply to provide source heat for the amplification reaction. The methods are performed at a temperature below 65° C., 60° C., 55° C., 50° C., 65° C., or 40° C. In some embodiments, the amplifying is performed at a temperature of 37° C. to 40° C. In some embodiments, the amplifying is performed at a temperature of 38° C.

The methods further comprise detecting an amplicon of the target DNA sequence. The detecting may comprise the use of a detection apparatus. Any detection apparatus that provides a readout that indicates whether a target nucleic acid is present in a sample may be used with the present invention. Detection devices may provide an analog or digital readout. The methods may also include the use of primers and reagents for detecting a control nucleic acid, i.e., a nucleic acid other than the target DNA sequence. Detection of the control nucleic acid may indicate that the method is working (i.e., a positive control) or may indicate that the method is producing non-specific results (i.e., a negative control).

In some embodiments, the amplicon is detected by lateral flow assay (LFA) using a lateral flow device. As used herein, a “lateral flow device” is a porous device capable of detecting the presence of a target nucleic acid traversing one or more beds. Lateral flow devices typically comprise (a) an aqueous sample loading area at one end; (b) an area comprising a capture agent comprising a first binding agent immobilized on a substrate; (c) an area comprising a reporter comprising a detectable label conjugated to a second binding agent, wherein the reporter is not bound to the lateral flow device and is capable of wicking across the lateral flow device; and (d) absorbent material, wherein the absorbent material wicks an aqueous sample across the lateral flow device when the aqueous sample is added to the sample loading area. The “binding agent” may be any moiety that binds to a specific 5′ tag. Suitable 5′ tag-binding agent pairs include, without limitation, ligand-protein pairs, antibody-antigen pairs, and antibody-hapten pairs. Thus, in some embodiments, the lateral flow device comprises a sample loading area, an amplification area, a solid support, an absorbent sample pad, or any combination thereof. A detailed description of exemplary lateral flow devices can be found in U.S. Patent Publication No. 2018/0148774, which is hereby incorporated by reference. The lateral flow device may be capable of multiplex nucleic acid detection (i.e., a testing device may comprise at least two, at least three, at least four, at least five, at least six, at least seven, at least eight, at least nine, at least ten, at least eleven, at least twelve, at least thirteen, at least fourteen, at least fifteen, at least twenty, or at least twenty five lateral flow devices).

In embodiments that utilize a lateral flow device, the assay results may be displayed using LFA strips that may provide a readout of one or more identifiable marks, such as a test or control line. The strips comprise a capture agent comprising a first binding agent immobilized on the lateral flow device in a region referred to as the “test area”. The test area can be any shape with well-defined boundaries, such as a dot or a line. The first binding agent binds to the 5′ tag of the amplicon-probe hybrid, capturing it in the test area. The first binding agent may be immobilized on the lateral flow device by covalent coupling or affinity binding. In the Examples, the inventors utilized an anti-FITC antibody as the first binding agent. The anti-FITC antibody was immobilized to the LFA strip such that binding of the FITC 5′ tag on the amplicon to the anti-FITC antibody captured the amplicon on the test line of the LFA strip. The LFA strip may comprise multiple test areas that are designed to capture different target nucleic acids for multiplex detection (e.g., specific to a different microorganism or virus).

In some embodiments, the detection device is configured such that detection is accomplished by visual inspection, either with or without additional instrumentation. For example, results can be quantified by imaging and analysis with a computer. The results can be scanned with a smartphone and electronically sent to a clinician, e.g., using an Adobe Acrobat grayscale converter or an ImageJ image processing software to quantify the visible light signal from a gold nanoparticle. Likewise, a color wheel for visualization of positive tests may be utilized.

In some embodiments, the devices further comprise a heating element, e.g., a heating element that is portable and does not require electricity. The heating element may comprise a battery-powered, cell phone-powered, or solar battery-powered heating film. Alternatively, the heating element may use a reversible or irreversible exothermic chemical reaction to generate heat.

An objective of the present invention is to provide rapid results indicating whether a patient is infected with a pathogen, allowing the patient to receive the results and treatment in a single visit. Thus, in some embodiments, the SNP in the target DNA sequence is detected in less than 60 minutes, preferably in less than 30 minutes.

In some embodiments, the methods can be used to determine if a patient is infected with a pathogen. In these embodiments, the target DNA sequence is specific to the pathogen such that detection of the amplicon following the RPA/RNaseHII assay indicates that the patient is infected with the pathogen. In addition, the disclosed methods may be used to determine if a patient is infected with a pathogen associated with antimicrobial resistance (AMR). In these embodiments, the SNP in the target DNA sequence is specifically associated with AMR, such that detection of the amplicon following the RPA/RNaseHII assay indicates that the patient is infected with a pathogen known to be resistant to a particular antimicrobial drug. If such a strain is detected, the antimicrobial treatment regimen for the patient can be managed accordingly (e.g., by treatment with an alternative antimicrobial drug).

### Devices

The present invention also provides point of need (PON) testing devices that utilize the disclosed RNaseHII/RPA compositions and methods. Such devices are known in the art, and include the devices disclosed in U.S. Patent Publication No. 2018/0148774 and PCT/US2018/063663, which are incorporated herein by this reference. The PON devices comprise a lateral flow device and the compositions of the present invention contained therein. In certain embodiments, the lateral flow device is a multiplexed lateral flow device to enable the detection of a multiplicity of target DNA sequences.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein are for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration of such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein is merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### Exemplary Embodiments

Embodiment 1. A composition for detecting a single nucleotide polymorphism (SNP) in a target DNA sequence comprising:
(a) a probe comprising a polynucleotide complementary to the target DNA sequence, the polynucleotide comprising in order from 5′ to 3′: a first DNA sequence, a RNA base complementary to the SNP, a second DNA sequence, and optionally a mismatched DNA base, wherein the polynucleotide is conjugated to a 5′ tag and a 3′ blocker;
(b) a forward primer;
(c) a reverse primer;
(d) a RNaseHII; and
(e) a recombinase polymerase amplification (RPA) reagent mix.
   Embodiment 2. The composition of embodiment 1, wherein the first DNA sequence has a length of 10 bases to 40 bases.
   Embodiment 3. The composition of embodiment 2, wherein the first DNA sequence has a length of 28 bases to 35 bases.
   Embodiment 4. The composition of any one of embodiments 1-3, wherein the second DNA sequence has a length of 4 bases to 10 bases.
   Embodiment The composition of embodiment 4, wherein the second DNA sequence has a length of 4-6 bases.
   Embodiment 6. The composition of any one of embodiments 1-5, wherein the 5′ tag is detectable by gel electrophoresis or lateral flow assay.
   Embodiment 7. The composition of any one of embodiments 1-6, wherein the 5′ tag is a fluorescein isothiocyanate tag.
   Embodiment 8. The composition of any one of embodiments 1-7, wherein the 3′ blocker is an inverted dT blocker.
   Embodiment 9. The composition of any one of embodiments 1-8, wherein the reverse primer comprises a biotin tag.
   Embodiment 10. The composition of any one of embodiments 1-9, wherein the RNaseHII selectively cleaves the probe at the 3′ side of the RNA base at an incubation temperature of 37 to 40° C.
   Embodiment 11. The composition of any one of embodiments 1-10, wherein the RNaseHII is a Pyrococcus abyssi RNaseHII, a Thermus thermophilus RNaseHII, a Chlamydia pneumoniae RNaseHII, or a Escherichia coli RNaseHII.
   Embodiment 12. The composition of any one of embodiments 1-11, wherein the RPA reaction mix comprises:
(a) a recombinase protein, optionally wherein the recombinase protein is T4 bacteriophage UvsX;
(b) a single-stranded binding protein, optionally wherein the single-stranded binding protein is gp32;
(c) a recombination mediator protein, optionally wherein the recombination mediator protein is T4 bacteriophage UvsY; and/or
(d) a strand-displacing polymerase, optionally wherein the strand-displacing polymerase is a large fragment of Bacillus subtilis polymerase I (Bsu).
   Embodiment 13. The composition of any one of embodiments 1-12, wherein the RPA reagent mix further comprises Triton-X 100, optionally wherein the Triton-X 100 is present in the composition at a concentration of about 0.00075% to about 0.01% further optionally wherein the Triton-X 100 is present in the composition at a concentration of about 0.006%.
   Embodiment 14. The composition of any one of embodiments 1-13, wherein the probe is present in the composition at a concentration of about 50 nM to about 500 nM, optionally wherein the probe is present in the composition at a concentration of about 250 nM.
   Embodiment 15. The composition of any one of embodiments 1-14, wherein the forward primer and/or the reverse primer is present in the composition at a concentration of about 100 nM to about 1000 nM, optionally wherein the forward primer and/or the reverse primer is present in the composition at a concentration of about 500 nM.
   Embodiment 16. A method for detecting a single nucleotide polymorphism (SNP) in a target DNA sequence:
(a) obtaining a sample suspected of comprising the target DNA sequence;
(b) contacting the target DNA sequence with the composition of any one of embodiments 1-16 to generate an amplification reaction mix;
(c) amplifying the target DNA sequence in the amplification reaction mix, wherein the amplifying is performed isothermally; and
(d) detecting an amplicon of the target DNA sequence.
   Embodiment 17. The method of embodiment 16, wherein the amplifying is performed at a temperature of 37 to 40° C.
   Embodiment 18. The method of embodiment 16 or 17, wherein the RNaseHII selectively cleaves the probe to generate a cleaved probe, and wherein the cleaved probe is an internal primer for the amplifying.
   Embodiment 19. The method of any one of embodiments 16-18, wherein the amplicon comprises the 5′ tag.
   Embodiment 20. The method of any one of embodiments 16-19, wherein a lysis agent is added to the sample prior to step (b).
   Embodiment 21. The method of any one of embodiments 16-20, wherein the sample is subjected to a DNA preprocessing step prior to step (b).
   Embodiment 22. The method of embodiment 21, wherein the DNA preprocessing step involves one or more of the following: shearing, sonication, nebulization, enzymatic digestion, reverse transcription, isolation, extraction, and purification.
   Embodiment 23. The method of any one of embodiments 16-19, wherein the sample is not subject to any further processing steps prior to or during the steps of the method.
   Embodiment 24. The method of any one of embodiments 16-23, wherein the detecting is performed by lateral flow assay.
   Embodiment 25. The method of any one of embodiments 16-24, wherein the sample is obtained from a subject.
   Embodiment 26. The method of embodiment 25, wherein detection of the target DNA sequence is indicative of the presence of a pathogen.
   Embodiment 27. The method of embodiment 26, wherein the pathogen is a bacterium, a fungus, a virus, or a protist.
   Embodiment 28. The method of embodiment 27, wherein the pathogen is a bacterium selected from the following: Neisseria gonorrhoeae, Salmonella enterica, Campylobacter jejuni, or Campylobacter coli.
   Embodiment 29. The method of any one of embodiments 26-28, wherein the SNP is associated with antimicrobial resistance.
   Embodiment 30. The method of embodiment 29, wherein the SNP is a penA gene mutation in Neisseria gonorrhoeae, an acrB mutation in Salmonella enterica, a gyrA gene mutation in Campylobacter jejuni, or a 23S rRNA gene mutation in Campylobacter coli.
   Embodiment 31. The method of any one of embodiments 16-30, wherein the method is performed in a point of need testing device.
   Embodiment 32. A probe for detecting a single nucleotide polymorphism (SNP) in a target DNA sequence comprising a polynucleotide complementary to the target DNA sequence, the polynucleotide comprising in order from 5′ to 3′:
(a) a first DNA sequence having a length of 10 bases to 40 bases;
(b) a RNA base complementary to the SNP;
(c) a second DNA sequence having a length of 4 bases to 10 bases; and
(d) optionally, a mismatched DNA base,
   wherein the polynucleotide is conjugated at the 5′ end to a tag and at the 3′ end to a blocker.
   Embodiment 33. The probe of embodiment 32, wherein the first DNA sequence has a length of 28 bases to 35 bases.
   Embodiment 34. The probe of embodiment 32 or 33, wherein the second DNA sequence has a length of 4-6 bases.
   Embodiment 35. The probe of any one of embodiments 32-34, wherein the tag is detectable by gel electrophoresis or lateral flow assay.
   Embodiment 36. The probe of any one of embodiments 32-35, wherein the tag is a fluorescein isothiocyanate tag.
   Embodiment 37. The probe of any one of embodiments 32-36, wherein the blocker is an inverted dT blocker.
   Embodiment 38. The probe of any one of embodiments 32-37, wherein the probe is configured to be selectively cleaved by a RNaseHII at the 3′ side of the SNP.

### Examples

While the compositions, methods, probes, and PON testing devices of the present invention may be utilized for diverse applications, the inventors exemplify the use for the detection of single nucleotide polymorphisms (SNPs) associated with antimicrobial resistance (AMR) in Neisseria gonorrhoeae, Salmonella enterica, and Campylobacter jejuni. By eliminating the need for costly conventional laboratory diagnostics, the present invention will enable to clinicians to quickly detect these AMR mutations, allowing for affordable screening, diagnosis, and treatment of bacterial illnesses.

To detect each specific SNP, an unmodified forward primer, a reverse primer tagged with biotin at the 5′ end, and a probe were designed. Each probe was designed to include a fluorescein isothiocyanate (FITC) tag at the 3-prime end, a RNA base (rA, rU, IC, or rG) in the middle complementary to the target SNP mutation, and a 3-prime end blocker. DNA samples were obtained from multiple bacterial strains having either the target SNP or the nucleotide present in the wild-type gene sequence (the “wild-type nucleotide”), amplified in a RNaseHII/RPA reaction mix, and analyzed by LFA and gel electrophoresis. The RNaseHII/RPA reaction mix included RNaseHII derived from either Pyrococcus abyssi or Escherichia coli. Finally, the ability of the present invention to detect SNPs was demonstrated in stool samples.

Neisseria gonorrhoeae

Three different probes were designed to detect the SNP mutations F505L, A511V, and A517G in the penA gene of Neisseria gonorrhoeae, respectively, and a universal forward and reverse primer for penA gene were also designed. The sequences for these primers and probes, along with the wild type penA and mutant penA target sequence are shown in Table 1.

TABLE 1
penA F505L, A511V, and A517G assay sequences and probes

| ID | Sequence 5′-3′ |
|---|---|
| penA | AGGTACGCAATCTGATGGTTTCCGTAACCGAG |
| Forward | (SEQ ID NO: 1) |
| penA | /5Biosg/AGTGGCTTGGTCGGGGAAATGCCCAAGATGTTC |
| Reverse | (SEQ ID NO: 2) |
| Probe | /56-FAM/ATGTCGGCGCTAAAACCGGTACGGCGCGCAAArCTGGT/3InvdT/ |
| F505L | (SEQ ID NO: 3) |
| Probe | /56-FAM/TACGGCGCGCAAACTGGTCAATGGCCGCTATGrUGGAC/3InvdT/ |
| A511V | (SEQ ID NO: 4) |
| Probe | /56-FAM/TCAATGGCCGCTATGTGGACAACAAACACGTCGrGTACG/3InvdT/ |
| A517G | (SEQ ID NO: 5) |
| Wild- | CGCGCGAGGTACGCAATCTGATGGTTTCCGTAACCGAGCCGGGCGGCAC |
| type | CGGTACGGCGGGTGCGGTGGACGGTTTCGATGTCGGCGCTAAAACCGGC |
| target | ACGGCGCGCAAGTTCGTCAACGGGCGTTATGCCGACAACAAACACGTCG |
| sequence | CTACCTTTATCGGTTTTGCCCCCGCCAAAAACCCCCGTGTGATTGTGGC |
| | GGTAACCATCGACGAACCGACTGCCCACGGCTATTACGGCGGCGTAGTG |
| | GCAGGGCCGCCCTTCAAAAAAATTATGGGCGGCAGCCTGAACATCTTGG |
| | GCATTTCCCCGACCAAGCCACTGACCGCCGCAGCCGTCAAAACACCGTC |
| | TTAA |
| | (SEQ ID NO: 6) |
| Mutant | CGCGCGAGGTACGCAATCTGATGGTTTCCGTAACCGAGCCGGGCGGCAC |
| target | CGGTACGGCGGGTGCGGTGGACGGTTTCGATGTCGGCGCTAAAACCGGT |
| sequence | ACGGCGCGCAAACTGGTCAATGGCCGCTATGTGGACAACAAACACGTC |
| | GGTACGTTTATCGGTTTTGCCCCCGCCAAAAACCCCCGTGTGATTGTGG |
| | CGGTAACCATCGACGAACCGACTGCCCACGGCTATTACGGCGGCGTAGT |
| | GGCAGGGCCGCCCTTCAAAAAAATTATGGGCGGCAGCCTGAACATCTTG |
| | GGCATTTCCCCGACCAAGCCACTGACCGCCGCAGCCGTCAAAACACCGT |
| | CTTAA |
| | (SEQ ID NO: 7) |

**/5Biosg/ is a biotin tag; /56-FAM/ is a fluorescein tag; rC, rU, rG, denote RNA bases; /3InvdT/ is an inverted dT blocker. SNP mutations are marked in the wild-type and mutant genome sequence by underlining and bolding. F505L mutation is TTC > CTG; A511V mutation is CCG > TGG; A517G mutation is CTA > GTA.

Each reaction mix contained an RPA mix from the TwisDx RPA basic kit, Pyrococcus abyssi RNaseHII (26.4 mU/μl; INTEGRATED DNA TECHNOLOGIES), Triton-X 100 (0.006%), the forward and reverse primers (500 nM each), the specific SNP probe (250 nM), and either wild-type DNA (2e4 cp/rxn) or target SNP DNA (200 cp/rxn). The wild-type Neisseria gonorrhoeae DNA was obtained from the strain designated ATCC 700825 DQ, and the target SNP DNA containing the AMR mutations was obtained from the strain designated ATCC BAA1846. The combined reaction mix was incubated at 38° C. for 30 min, and the amplicons were analyzed using LFA strips and gel electrophoresis.

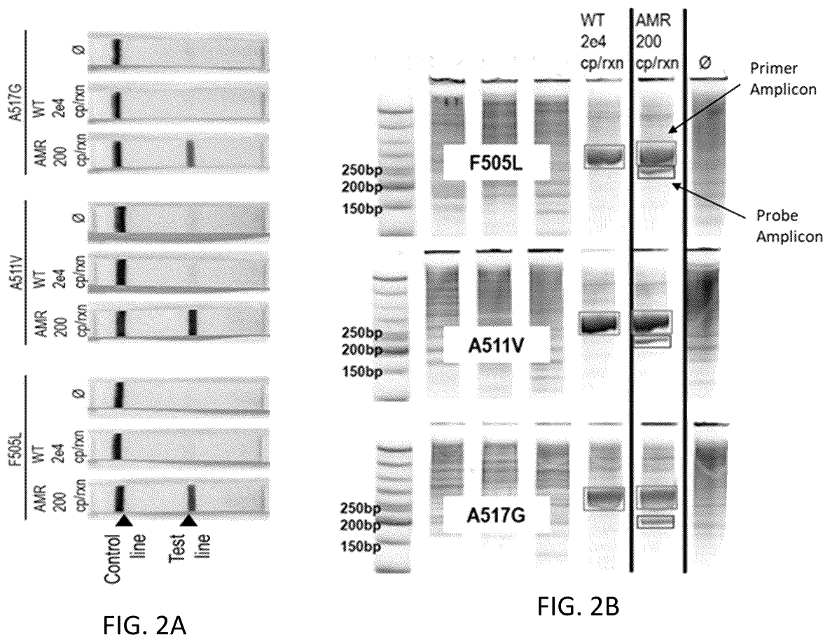

The LFA results demonstrated specific detection of the F505L, A511V, and A517G SNP mutations (FIG. 2A). In addition, gel electrophoresis confirmed this selective amplification, as the gel images indicated the presence of primer amplicons in both the wild-type samples and target SNP samples, while the probe amplicons were present only in the target SNP samples (FIG. 2B).

Salmonella enterica

Two different probes were designed to detect two different point mutations (R717L, R717Q) in Salmonella enterica acrB gene, and a universal forward and reverse primer for acrB gene were also designed. The sequences for these primers and probes, along with the wild type acrB and mutant acrB target sequence are shown in Table 2.

TABLE 2
acrB R717Q and R717L assay sequences and probes

| ID | Sequence 5′-3′ |
|---|---|
| acrB Forward | AAAACTCACCCAGGCRCGTAATCAGTTGTTC |
| | (SEQ ID NO: 8) |
| acrB Reverse | /5Biosg/TTCGGACATCACGTAAACTTTCTTCACACGAC |
| | (SEQ ID NO: 9) |
| Probe R717L | /56-FAM/CGAAATATCCTGATCTGCTGGTAGGCGTTCrUACCTG/3InvdT/ |
| | (SEQ ID NO: 10) |
| Probe R717Q | /56-FAM/CGAAATATCCTGATCTGCTGGTCGGCGTTCrAACCTG/3InvdT/ |
| | (SEQ ID NO: 11) |
| Wild-type | CTTTGACTTCGAGTTGATTGACCAGGCGGGACTTGGTCATGAAAAAC |
| target | TCACCCAGGCACGTAATCAGTTGTTCGGCGAGGTGGCGAAATATCCT |
| sequence | GATCTGCTGGTCGGCGTTCGACCTAACGGTCTGGAAGATACGCCGCA |
| | GTTTAAAATCGATATCGACCAGGAAAAAGCTCAGGCGCTGGGCGTAT |
| | CTATTAGCGACATTAATACCACGCTGGGCGCAGCATGGGGCGGCAGC |
| | TATGTAAACGACTTTATCGATCGCGGTCGTGTGAAGAAAGTTTACGTG |
| | ATGTCCGAAGCGAAATACCGCATGTTGCCGGATGATATTAAC |
| | (SEQ ID NO: 12) |
| Mutant target | CTTTGACTTCGAGTTGATTGACCAGGCGGGACTTGGTCATGAAAAAC |
| sequence | TCACCCAGGCGCGTAATCAGTTGTTCGGCGAAGTGGCGAAATATCCT |
| (717L) | GATCTGCTGGTAGGCGTTCTACCTAACGGCCTGGAAGATACGCCGCA |
| | GTTTAAAATCGATATCGACCAGGAAAAAGCTCAGGCGCTGGGCGTGT |
| | CTATTAGCGACATTAATACCACGCTGGGCGCAGCATGGGGCGGCAGC |
| | TATGTAAACGACTTTATCGATCGCGGTCGTGTGAAGAAAGTTTACGTG |
| | ATGTCCGAAGCGAAATACCGCATGTTGCCGGATGATATTAACG |
| | (SEQ ID NO: 13) |
| Mutant target | CTTTGACTTCGAGTTGATTGACCAGGCGGGACTTGGTCATGAAAAAC |
| sequence | TCACCCAGGCACGTAATCAGTTGTTCGGCGAGGTGGCGAAATATCCT |
| (717Q) | GATCTGCTGGTCGGCGTTCAACCTAACGGTCTGGAAGATACGCCGCA |
| | GTTTAAAATCGATATCGACCAGGAAAAAGCTCAGGCGCTGGGCGTAT |
| | CTATTAGCGACATTAATACCACGCTGGGCGCAGCATGGGGCGGCAGC |
| | TATGTAAACGACTTTATCGATCGCGGTCGTGTGAAGAAAGTTTACGTG |
| | ATGTCCGAAGCGAAATACCGCATGTTGCCGGATGATATTAACG |
| | (SEQ ID NO: 14) |

**/5Biosg/ is a biotin tag; /56-FAM/ is a fluorescein tag; rU and rA denotes RNA bases; /3 InvdT/ is an inverted dT blocker. R = G/A. The SNP mutations are marked in the wild-type and mutant genome sequence by underlining and bolding. R717L mutation is CGA > CTA; R717Q mutation is CGA > CAA.

Each reaction mix contained an RPA mix from the TwisDx RPA basic kit, Pyrococcus abyssi RNaseHII (26.4 mU/μl; INTEGRATED DNA TECHNOLOGIES), Triton-X 100 (0.006%), the forward and reverse primers (500 nM each), the specific SNP probe (250 nM), and either R717L target SNP DNA (4.26 pg or 42.6 pg) or R717Q target SNP DNA (4.11 pg or 41.1 pg). The R717L target SNP DNA was obtained from the strain designated PNUSAS290791, and the target R717Q target SNP DNA was obtained from the strain designated PNUSAS193042. These strains were collected, cultured, sequenced, and tested to be resistant to azithromycin by the Iowa State Hygienic Lab at the University of Iowa. The combined reaction mix was incubated at 38° C. for 30 min, and the amplicons were analyzed using LFA strips and gel electrophoresis.

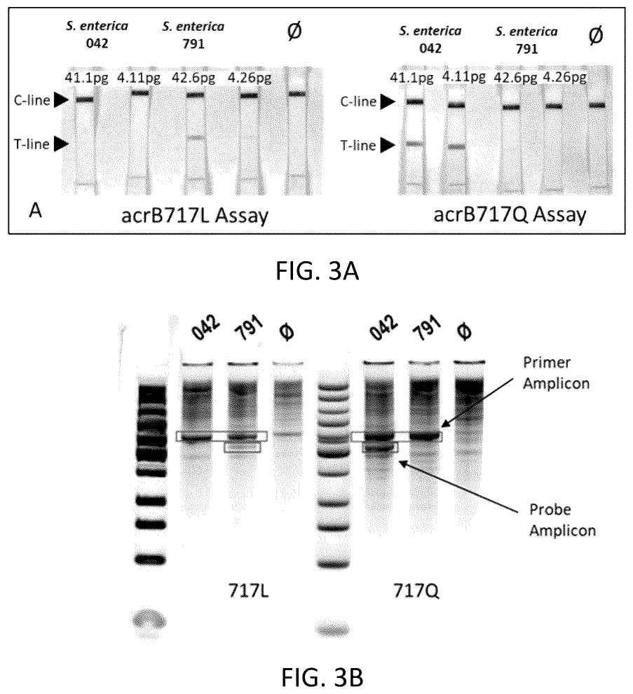

The specificity of each target probe was examined by conducting four separate RPA/RNaseHII assays using either R717L target SNP DNA (4.26 pg or 42.6 pg) or R717Q target SNP DNA (4.11 pg or 41.1 pg). The LFA results showed specific detection of the R717L and R717Q SNP mutations by their respective probes at both tested DNA concentrations, demonstrating the ability to differentiate between two different SNPs located at the same nucleotide (FIG. 3A). In addition, gel electrophoresis confirmed this selective amplification, as the amplicons for each probe were present only in the samples containing its designated target SNP (FIG. 3B).

Campylobacter jejuni

One probe was designed to detect the SNP mutation T86I (C257T) in the gyrA gene of Campylobacter jejuni, and a universal forward and reverse primer for the gyrA gene were also designed. The sequences for these primers and probes, along with the wild type gyrA and mutant gyrA target sequence are shown in Table 3.

TABLE 3
gyrA T86I (C257T) assay sequences and probes

| ID | Sequence 5′-3′ |
|---|---|
| gyrA | AGATCCAAAGTTGCCTTGTCCTGTAATACTTGG |
| Forward | (SEQ ID NO: 15) |
| gyrA | /5Biosg/TGACGCAAGAGATGGTTTAAAGCCTGTTCATAG |
| Reverse | (SEQ ID NO: 16) |
| Probe | /56-FAM/GCCATTCTAACCAAAGCATCATAAACTGCTrATATCA/3InvdT/ |
| T86I | (SEQ ID NO: 17) |
| (C257T) | |
| Wild- | GGTAGTTACTTAGACTATTCTATGAGTGTTATTATAGGTCGTGCTTTGCCT |
| type | GACGCAAGAGATGGTTTAAAGCCTGTTCATAGAAGAATTTTATATGCTAT |
| target | GCAAAATGATGAGGCAAAAAGTAGAACAGATTTTGTCAAATCAGCCCG |
| sequence | TATAGTGGGTGCTGTTATAGGTCGTTATCATCCACATGGAGATACAGCAG |
| | TTTATGATGCTTTGGTTAGAATGGCTCAAGATTTTTCTATGAGATATCCAA |
| | GTATTACAGGACAAGGCAACTTTGGATCTATAGATGGTGATAGCGCTGCT |
| | GCGATGCGTTATACTGAAGCAAAAAT |
| | (SEQ ID NO: 18) |
| Mutant | GGTAGTTACTTAGACTATTCTATGAGTGTTATTATAGGTCGTGCTTTGCCT |
| target | GACGCAAGAGATGGTTTAAAGCCTGTTCATAGAAGAATTTTATATGCTAT |
| sequence | GCAAAATGATGAGGCAAAAAGTAGAACAGATTTTGTCAAATCAGCCCG |
| | TATAGTGGGTGCTGTTATAGGTCGTTATCATCCACATGGAGATATAGCAG |
| | TTTATGATGCTTTGGTTAGAATGGCTCAAGATTTTTCTATGAGATATCCAA |
| | GTATTACAGGACAAGGCAACTTTGGATCTATAGATGGTGATAGCGCTGCT |
| | GCGATGCGTTATACTGAAGCAAAAAT |
| | (SEQ ID NO: 19) |

**/5Biosg/ is a biotin tag; /56-FAM/ is a fluorescein tag; rA denotes RNA bases; /3InvdT/ is an inverted dT blocker. The SNP mutations are marked in the wild-type and mutant genome sequence by underlining and bolding.

Each reaction mix contained an RPA mix from the TwisDx RPA basic kit, Pyrococcus abyssi RNascHII (26.4 mU/μl; INTEGRATED DNA TECHNOLOGIES), Triton-X 100 (0.006%), the forward and reverse primers (500 nM each), the specific SNP probe (250 nM), and either wild-type DNA or target SNP DNA. The wild-type Campylobacter DNA was obtained from the Campylobacter jejuni strain CIP 702 and the Campylobacter coli strain LRA 069.05.89 from ATCC. The target SNP DNA was obtained from Campylobacter jejuni strain D4344 from ATCC. The combined reaction mix was incubated at 38° C. for 30 min, and the amplicons were analyzed using LFA strips and gel electrophoresis.

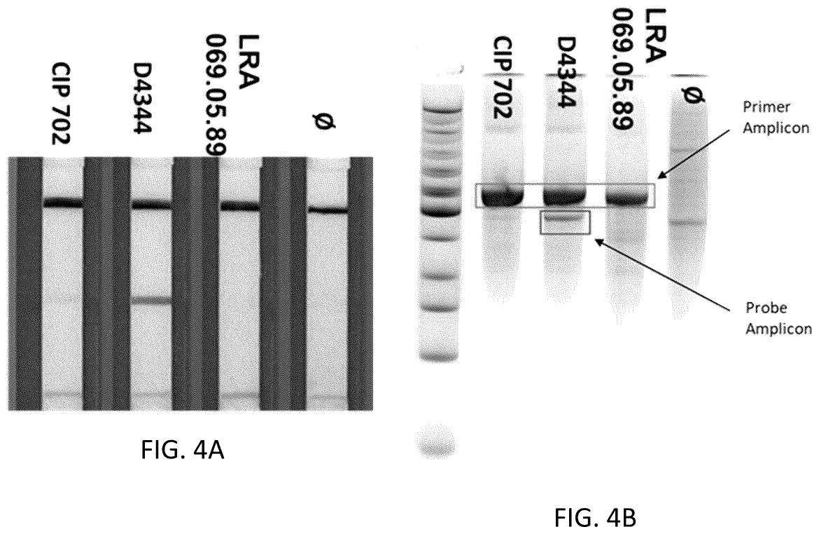

The LFA results demonstrated specific detection of the T861 (C257T) SNP mutation (FIG. 4A). In addition, gel electrophoresis confirmed this selective amplification, as the amplicons for the probe were present only in the samples containing the T86I (C257T) target SNP (FIG. 4B).

In addition, the ability of the primers and probes shown in Table 3 to detect the SNP mutation T86I (C257T) in the gyrA gene of Campylobacter jejuni was also assessed using a different RNaseHII, derived from Escherichia coli. Each reaction mix contained an RPA mix from the TwisDx RPA basic kit, Escherichia coli RNascHII (125 mU/μl; NEW ENGLAND BIOLABS), Triton-X 100 (0.006%), forward and reverse primers (500 nM each), the specific SNP probe (250 nM), and either wild-type DNA or target SNP DNA. The wild-type and target DNA were presented in a sample at two different concentrations: 2×10⁴ CFU and 2,000 CFU. The wild-type Campylobacter DNA was obtained from the Campylobacter jejuni strain CIP 702, and the target SNP DNA was obtained from Campylobacter jejuni strain D4344 from ATCC. The combined reaction mix was incubated at 38° C. for 60 min, and the amplicons were analyzed using LFA strips and gel electrophoresis.

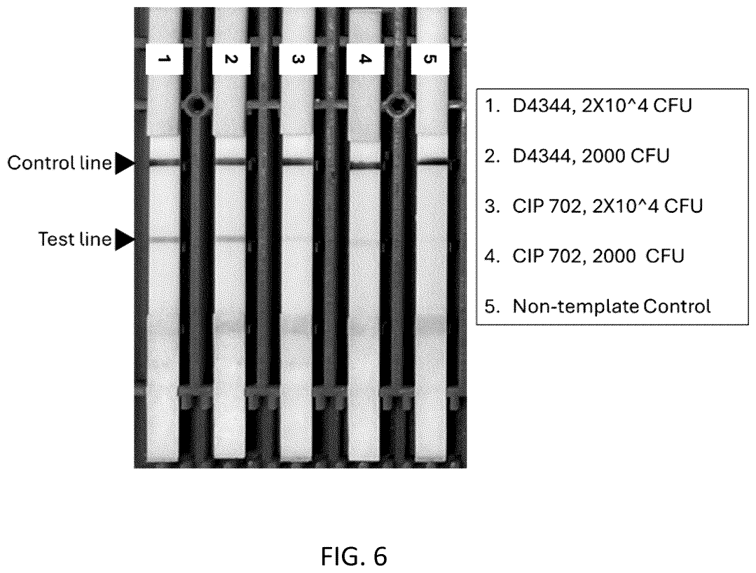

The LFA results demonstrated that the SNP mutation T86I (C257T) in gyrA can also be detected using Escherichia coli RNaseHII (FIG. 6). The LFA results indicate that E. coli RNaseHII preferentially cleaves the RNA base that is complementary to the target SNP. Specifically, a stronger test line signal was produced in the mutant samples than in the wild-type samples, even when a high copy number (2×10\hat{ }4 CFU) of wild-type sample was presented. In addition, gel electrophoresis confirmed this selective amplification, as the amplicons for the probe were present only in the samples containing the T86I (C257T) target SNP.

Campylobacter coli

One probe was designed to detect the SNP mutation A2075G in 23S rRNA gene of Campylobacter, and a universal forward and reverse primers for the 23S rRNA gene were also designed. The sequences for these primers and probes, along with the wild-type 23S rRNA and mutant 23S rRNA target sequence are shown in Table 4.

TABLE 4
23S rRNA A2075G assay sequences and probes

| ID | Sequence 5′-3′ |
|---|---|
| 23S rRNA | GGGTTAGCATTAGCGAAGCTCTTGATCGAAGCC |
| Forward | (SEQ ID NO: 20) |
| 23S rRNA | /5Biosg/CTCAACAATGGCTCATATACAACTGGCGTCAT |
| Reverse | (SEQ ID NO: 21) |
| Probe | /56-FAM/GAAAATTCCTCCTACCCGCGGCAAGACGGArGAGACT/3InvdT/ |
| A2075G | (SEQ ID NO: 22) |
| Wild-type | CTGCCCGGTGCTCGAAGGTTAATTGATGGGGTTAGCATTAGCGAAGCT |
| target | CTTGATCGAAGCCCGAGTAAACGGCGGCCGTAACTATAACGGTCCTA |
| sequence | AGGTAGCGAAATTCCTTGTCGGTTAAATACCGACCTGCATGAATGGCG |
| | TAACGAGATGGGAGCTGTCTCAAAGAGGGATCCAGTGAAATTGTAGT |
| | GGAGGTGAAAATTCCTCCTACCCGCGGCAAGACGGAAAGACCCCGTG |
| | GACCTTTACTACAGCTTGACACTGCTACTTGGATAAGAATGTGCAGGA |
| | TAGGTGGGAGGCTTTGAGTATATGACGCCAGTTGTATATGAGCCATTG |
| | TTGAGATACCACTCTTTCTTATTTGGGTAGCTAACCAGCTTGAG |
| | (SEQ ID NO: 23) |
| Mutant | CTGCCCGGTGCTCGAAGGTTAATTGATGGGGTTAGCATTAGCGAAGCT |
| target | CTTGATCGAAGCCCGAGTAAACGGCGGCCGTAACTATAACGGTCCTA |
| sequence | AGGTAGCGAAATTCCTTGTCGGTTAAATACCGACCTGCATGAATGGCG |
| | TAACGAGATGGGAGCTGTCTCAAAGAGGGATCCAGTGAAATTGTAGT |
| | GGAGGTGAAAATTCCTCCTACCCGCGGCAAGACGGAGAGACCCCGTG |
| | GACCTTTACTACAGCTTGACACTGCTACTTGGATAAGAATGTGCAGGA |
| | TAGGTGGGAGGCTTTGAGTATATGACGCCAGTTGTATATGAGCCATTG |
| | TTGAGATACCACTCTTTCTTATTTGGGTAGCTAACCAGCTTGAG |
| | (SEQ ID NO: 24) |

**/5Biosg/ is a biotin tag; /56-FAM/ is a fluorescein tag; rG denotes RNA bases; /3InvdT/ is an inverted dT blocker. The SNP mutations are marked in the wild-type and mutant genome sequence by underlining and bolding.

Each reaction mix contained an RPA mix from the TwisDx RPA basic kit, Pyrococcus abyssi RNaseHII (6.6 mU/μl; INTEGRATED DNA TECHNOLOGIES), Triton-X 100 (0.003%), the forward and reverse primers (500 nM each), the specific SNP probe (250 nM), and either wild-type DNA or target SNP DNA. The wild-type Campylobacter DNA was obtained from the Campylobacter jejuni strain CIP 702 and the Campylobacter jejuni strain D4344 from ATCC, and the target SNP DNA containing the A2075G SNP mutation was obtained from the Campylobacter coli strain LRA 069.05.89 from ATCC. The combined reaction mix was incubated at 38° C. for 30 min, and the amplicons were analyzed using LFA strips and gel electrophoresis.

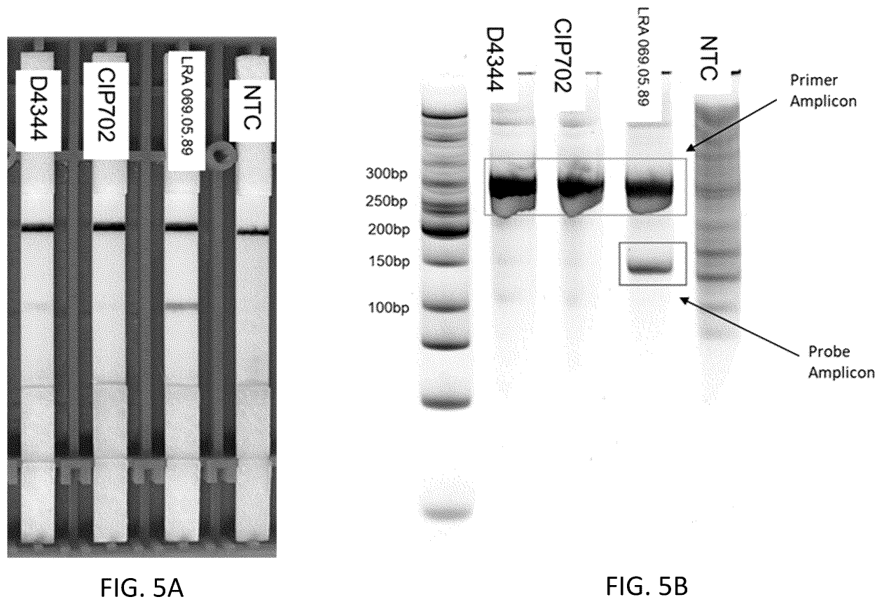

The LFA results demonstrated specific detection of the A2075G SNP mutation (FIG. 5A). In addition, gel electrophoresis confirmed this selective amplification, as the amplicons for the probe was present only in the Campylobacter coli sample containing the T86I (C257T) target SNP (FIG. 5B).

### Stool Sample Analysis

The ability of the present compositions and methods to detect the Salmonella enterica acrB gene SNP mutation R717Q and the Campylobacter jejuni gyrA gene SNP mutation T86I (C257T) was also assessed using stool samples.

20 negative stool samples purchased from Discover Life Sciences were aliquoted for spike-in. For Salmonella SNP assay, 5 wild-type samples (acrB717R) were made by spiking in Salmonella isolate PNUSAS247742 at 3000 CFU per swab, 12 mutant samples (acrB717Q) were made by spiking in Salmonella isolate PNUSAS193042 at 3000 or 10000 CFU per swab, and 24 aliquots were tested as negative control samples. For the Campylobacter SNP assay, 4 wild-type samples (gyrA) were made by spiking in Campylobacter strain CIP702 at 3000 or 10000 CFU per swab, 10 mutant samples (gyrA T86I) were made by spiking in Campylobacter strain D4344 at 10000 or 15000 CFU per swab, and 19 aliquots were tested as negative control samples.

To perform the SNP detection assay, one swab of the spiked stool sample was mixed with 1 ml of bind/wash buffer (1×PBS, 1% BSA, 0.05% Tween20, 0.05% NaN3) to elute the sample. The eluate was transferred to 1 mg of E coli. antibody (Ab)-conjugated magnetic beads and incubated on rotation for 5 min to clean up the background E. coli. After the cleanup, the eluate was further transferred to 1 mg Ab-conjugated magnetic beads of either Salmonella antibody or Campylobacter antibody to enrich the targeted bacteria. After 30 min rotation/incubation, the beads were washed three times using the bind/wash buffer, and heat lysed at 95° C. for 15 min in 100 μl 10 mM TrisHCl buffer, pH 8.0 containing 14 mM magnesium acetate. The lysate was added to lyophilized RPA assays containing 26.4 mU/μl Pyrococcus abyssi RNaseHII from INTEGRATED DNA TECHNOLOGIES and 0.006% Triton-X 100, and further containing either the acrB R717Q assay or gyrA T86I (C257T) assay primers and probes shown in Table 2 and Table 3, respectively. The rehydrated assays were incubated at 38° C. for 30 min and the results were read with LFA assay strips.

As shown in Table 5, the Salmonella SNP acrB R717Q was detected with 100% sensitivity and 100% specificity against wild-type Salmonella and other bacteria occurring in stool samples. In addition, the Campylobacter SNP gyrA T86I (C257T) was detected with 90.0% sensitivity and with 90.9% specificity against wild-type Campylobacter and other bacteria occurring in stool samples. This result indicates the described invention can be used to detect SNP-carrying bacteria from clinical stool samples.

TABLE 5
SNP detection in stool samples.

| | True | True | False | False | | | 95% CI | 95% CI |
|---|---|---|---|---|---|---|---|---|
| Assay | Positives | Negatives | Positives | Negatives | Sensitivity | Specificity | Sensitivity | specificity |
| acrB | 12 | 28 | 0 | 0 | 100% | 100% | 73.54% to | 87.66% to |
| R717Q | | | | | | | 100.00% | 100.00% |
| gyrA | 9 | 20 | 2 | 1 | 90.0% | 90.9% | 55.50% to | 70.84% to |
| T86I | | | | | | | 99.75% | 98.88% |

### REFERENCES

1. RECOMBINASE POLYMERASE AMPLIFICATION, US Patent Pub. No.: 2019/0360030 A1
2. METHODS AND COMPOSITIONS FOR NUCLEIC ACID AMPLIFICATION, US Patent Pub. No.: US 2019/0284597 A1
3. Dobosy, Joseph R., et al. “RNase H-dependent PCR (rhPCR): improved specificity and single nucleotide polymorphism detection using blocked cleavable primers.” BMC biotechnology 11 (2011): 1-18.
4. Beltz, Kristin, et al. “A high-performing and cost-effective SNP genotyping method using rhPCR and universal reporters.” Advances in Bioscience and Biotechnology 9.09 (2018): 497.
5. DNA POLYMERASE MUTANTS WITH INCREASED TEMPLATE DISCRIMINATION ACTIVITY, JP7266645B2

1. A composition for detecting a single nucleotide polymorphism (SNP) in a target DNA sequence comprising:
(a) a probe comprising a polynucleotide complementary to the target DNA sequence, the polynucleotide comprising in order from 5′ to 3′: a first DNA sequence, a RNA base complementary to the SNP, a second DNA sequence, and optionally a mismatched DNA base, wherein the polynucleotide is conjugated to a 5′ tag and a 3′ blocker;
(b) a forward primer;
(c) a reverse primer;
(d) a RNaseHII; and
(e) a recombinase polymerase amplification (RPA) reagent mix.

2. The composition of claim 1, wherein the first DNA sequence has a length of 10 bases to 40 bases.

3. The composition of claim 1, wherein the second DNA sequence has a length of 4 bases to 10 bases.

4. The composition of claim 1, wherein the 5′ tag is detectable by gel electrophoresis or lateral flow assay.

5. The composition of claim 1, wherein the 5′ tag is a fluorescein isothiocyanate tag.

6. The composition of claim 1, wherein the 3′ blocker is an inverted dT blocker.

7. The composition of claim 1, wherein the reverse primer comprises a biotin tag.

8. The composition of claim 1, wherein the RNaseHII selectively cleaves the probe at the 3′ side of the RNA base at an incubation temperature of 37 to 40° C.

9. The composition of claim 1, wherein the RNaseHII is a Pyrococcus abyssi RNaseHII, a Thermus thermophilus RNaseHII, a Chlamydia pneumoniae RNaseHII, or a Escherichia coli RNaseHII.

10. The composition of claim 1, wherein the RPA reaction mix comprises:
(a) a recombinase protein, optionally wherein the recombinase protein is T4 bacteriophage UvsX;
(b) a single-stranded binding protein, optionally wherein the single-stranded binding protein is gp32;
(c) a recombination mediator protein, optionally wherein the recombination mediator protein is T4 bacteriophage UvsY; and/or
(d) a strand-displacing polymerase, optionally wherein the strand-displacing polymerase is a large fragment of Bacillus subtilis polymerase I (Bsu).

11. A method for detecting a single nucleotide polymorphism (SNP) in a target DNA sequence:
(a) obtaining a sample suspected of comprising the target DNA sequence;
(b) contacting the target DNA sequence with the composition of claim 1 to generate an amplification reaction mix;
(c) amplifying the target DNA sequence in the amplification reaction mix, wherein the amplifying is performed isothermally; and
(d) detecting an amplicon of the target DNA sequence.

12. The method of claim 11, wherein the amplifying is performed at a temperature of 37 to 40° C.

13. The method of claim 11, wherein the probe hybridizes to the target DNA sequence.

14. The method of claim 13, wherein following hybridization of the probe to the target DNA sequence, the RNaseHII selectively cleaves the probe at the 3′ side of the RNA base to generate a cleaved probe, and wherein the cleaved probe is an internal primer for the amplifying.

15. The method of claim 11, wherein the amplicon comprises the 5′ tag.

16. The method of claim 11, wherein the detecting is performed by lateral flow assay.

17. The method of claim 11, wherein the sample is obtained from a subject, and/or wherein detection of the target DNA sequence is indicative of the presence of a pathogen.

18. The method of claim 17, wherein the SNP is associated with antimicrobial resistance.

19. A probe for detecting a single nucleotide polymorphism (SNP) in a target DNA sequence comprising a polynucleotide complementary to the target DNA sequence, the polynucleotide comprising in order from 5′ to 3′:
(a) a first DNA sequence having a length of 10 bases to 40 bases;
(b) a RNA base complementary to the SNP;
(c) a second DNA sequence having a length of 4 bases to 10 bases; and
wherein the polynucleotide is conjugated at the 5′ end to a tag and at the 3′ end to a blocker.

20. The probe of claim 19, wherein (i) the probe further comprises a mismatched DNA base, (ii) the first DNA sequence has a length of 28 bases to 35 bases, (iii) the second DNA sequence has a length of 4-6 bases, and/or (iv) the probe is configured to be selectively cleaved by a RNaseHII at the 3′ side of the SNP.


SEQUENCE LISTING (24 sequences)

SEQ ID NO: 1 - DNA, 32 bp, synthetic construct
  source 1..32: mol_type = other DNA
aggtacgcaatctgatggtttccgtaaccgag

SEQ ID NO: 2 - DNA, 33 bp, synthetic construct
  source 1..33: mol_type = other DNA
agtggcttggtcggggaaatgcccaagatgttc

SEQ ID NO: 3 - DNA, 38 bp, synthetic construct
  source 1..38: mol_type = other DNA
  misc_feature 32..33: note = RNA
  misc_feature 1..31: note = DNA
  misc_feature 34..38: note = DNA
  misc_feature 38: note = 3 prime-3 prime phosphodiester bond to 3 prime-inverted thymidine
atgtcggcgctaaaaccggtacggcgcgcaaarctggt

SEQ ID NO: 4 - DNA, 37 bp, synthetic construct
  source 1..37: mol_type = other DNA
  misc_feature 32: note = RNA
  misc_feature 1..31: note = DNA
  misc_feature 33..37: note = DNA
  misc_feature 37: note = 3 prime-3 prime phosphodiester bond to 3 prime-inverted thymidine
tacggcgcgcaaactggtcaatggccgctatgtggac

SEQ ID NO: 5 - DNA, 37 bp, synthetic construct
  source 1..37: mol_type = other DNA
  misc_feature 33: note = RNA
  misc_feature 1..32: note = DNA
  misc_feature 33..37: note = DNA
  misc_feature 37: note = 3 prime-3 prime phosphodiester bond to 3 prime-inverted thymidine
tcaatggccgctatgtggacaacaaacacgtcgtacg

SEQ ID NO: 6 - DNA, 347 bp, Neisseria gonorrhoeae
  source 1..347: mol_type = genomic DNA
cgcgcgaggtacgcaatctgatggtttccgtaaccgagccgggcggcaccggtacggcgggtgcggtggacggtttcgatgtcggcgctaaaaccggcacggcgcgcaagttcgtcaacgggcgttatgccgacaacaaacacgtcgctacctttatcggttttgcccccgccaaaaacccccgtgtgattgtggcggtaaccatcgacgaaccgactgcccacggctattacggcggcgtagtggcagggccgcccttcaaaaaaattatgggcggcagcctgaacatcttgggcatttccccgaccaagccactgaccgccgcagccgtcaaaacaccgtcttaa

SEQ ID NO: 7 - DNA, 347 bp, synthetic construct
  source 1..347: mol_type = other DNA
cgcgcgaggtacgcaatctgatggtttccgtaaccgagccgggcggcaccggtacggcgggtgcggtggacggtttcgatgtcggcgctaaaaccggtacggcgcgcaaactggtcaatggccgctatgtggacaacaaacacgtcggtacgtttatcggttttgcccccgccaaaaacccccgtgtgattgtggcggtaaccatcgacgaaccgactgcccacggctattacggcggcgtagtggcagggccgcccttcaaaaaaattatgggcggcagcctgaacatcttgggcatttccccgaccaagccactgaccgccgcagccgtcaaaacaccgtcttaa

SEQ ID NO: 8 - DNA, 31 bp, synthetic construct
  source 1..31: mol_type = other DNA
aaaactcacccaggcrcgtaatcagttgttc

SEQ ID NO: 9 - DNA, 32 bp, synthetic construct
  source 1..32: mol_type = other DNA
ttcggacatcacgtaaactttcttcacacgac

SEQ ID NO: 10 - DNA, 36 bp, synthetic construct
  source 1..36: mol_type = other DNA
  misc_feature 30..31: note = RNA
  misc_feature 1..29: note = DNA
  misc_feature 32..36: note = DNA
  misc_feature 36: note = 3 prime-3 prime phosphodiester bond to 3 prime-inverted thymidine
cgaaatatcctgatctgctggtaggcgttctacctg

SEQ ID NO: 11 - DNA, 36 bp, synthetic construct
  source 1..36: mol_type = other DNA
  misc_feature 30: note = RNA
  misc_feature 1..29: note = DNA
  misc_feature 31..36: note = DNA
  misc_feature 36: note = 3 prime-3 prime phosphodiester bond to 3 prime-inverted thymidine
cgaaatatcctgatctgctggtcggcgttcaacctg

SEQ ID NO: 12 - DNA, 325 bp, Salmonella enterica
  source 1..325: mol_type = genomic DNA
ctttgacttcgagttgattgaccaggcgggacttggtcatgaaaaactcacccaggcacgtaatcagttgttcggcgaggtggcgaaatatcctgatctgctggtcggcgttcgacctaacggtctggaagatacgccgcagtttaaaatcgatatcgaccaggaaaaagctcaggcgctgggcgtatctattagcgacattaataccacgctgggcgcagcatggggcggcagctatgtaaacgactttatcgatcgcggtcgtgtgaagaaagtttacgtgatgtccgaagcgaaataccgcatgttgccggatgatattaac

SEQ ID NO: 13 - DNA, 326 bp, synthetic construct
  source 1..326: mol_type = other DNA
ctttgacttcgagttgattgaccaggcgggacttggtcatgaaaaactcacccaggcgcgtaatcagttgttcggcgaagtggcgaaatatcctgatctgctggtaggcgttctacctaacggcctggaagatacgccgcagtttaaaatcgatatcgaccaggaaaaagctcaggcgctgggcgtgtctattagcgacattaataccacgctgggcgcagcatggggcggcagctatgtaaacgactttatcgatcgcggtcgtgtgaagaaagtttacgtgatgtccgaagcgaaataccgcatgttgccggatgatattaacg

SEQ ID NO: 14 - DNA, 326 bp, synthetic construct
  source 1..326: mol_type = other DNA
ctttgacttcgagttgattgaccaggcgggacttggtcatgaaaaactcacccaggcacgtaatcagttgttcggcgaggtggcgaaatatcctgatctgctggtcggcgttcaacctaacggtctggaagatacgccgcagtttaaaatcgatatcgaccaggaaaaagctcaggcgctgggcgtatctattagcgacattaataccacgctgggcgcagcatggggcggcagctatgtaaacgactttatcgatcgcggtcgtgtgaagaaagtttacgtgatgtccgaagcgaaataccgcatgttgccggatgatattaacg

SEQ ID NO: 15 - DNA, 33 bp, synthetic construct
  source 1..33: mol_type = other DNA
agatccaaagttgccttgtcctgtaatacttgg

SEQ ID NO: 16 - DNA, 33 bp, synthetic construct
  source 1..33: mol_type = other DNA
tgacgcaagagatggtttaaagcctgttcatag

SEQ ID NO: 17 - DNA, 36 bp, synthetic construct
  source 1..36: mol_type = other DNA
  misc_feature 30: note = RNA
  misc_feature 1..29: note = DNA
  misc_feature 31..36: note = DNA
  misc_feature 36: note = 3 prime-3 prime phosphodiester bond to 3 prime-inverted thymidine
gccattctaaccaaagcatcataaactgctatatca

SEQ ID NO: 18 - DNA, 326 bp, Campylobacter jejuni
  source 1..326: mol_type = genomic DNA
ggtagttacttagactattctatgagtgttattataggtcgtgctttgcctgacgcaagagatggtttaaagcctgttcatagaagaattttatatgctatgcaaaatgatgaggcaaaaagtagaacagattttgtcaaatcagcccgtatagtgggtgctgttataggtcgttatcatccacatggagatacagcagtttatgatgctttggttagaatggctcaagatttttctatgagatatccaagtattacaggacaaggcaactttggatctatagatggtgatagcgctgctgcgatgcgttatactgaagcaaaaat

SEQ ID NO: 19 - DNA, 326 bp, synthetic construct
  source 1..326: mol_type = other DNA
ggtagttacttagactattctatgagtgttattataggtcgtgctttgcctgacgcaagagatggtttaaagcctgttcatagaagaattttatatgctatgcaaaatgatgaggcaaaaagtagaacagattttgtcaaatcagcccgtatagtgggtgctgttataggtcgttatcatccacatggagatatagcagtttatgatgctttggttagaatggctcaagatttttctatgagatatccaagtattacaggacaaggcaactttggatctatagatggtgatagcgctgctgcgatgcgttatactgaagcaaaaat

SEQ ID NO: 20 - DNA, 33 bp, synthetic construct
  source 1..33: mol_type = other DNA
gggttagcattagcgaagctcttgatcgaagcc

SEQ ID NO: 21 - DNA, 31 bp, synthetic construct
  source 1..31: mol_type = other DNA
tcaacaatggctcatatacaactggcgtcat

SEQ ID NO: 22 - DNA, 36 bp, synthetic construct
  misc_feature 30: note = RNA
  misc_feature 1..29: note = DNA
  misc_feature 31..36: note = DNA
  source 1..36: mol_type = other DNA
  misc_feature 36: note = 3 prime-3 prime phosphodiester bond to 3 prime-inverted thymidine
gaaaattcctcctacccgcggcaagacggagagact

SEQ ID NO: 23 - DNA, 377 bp, Campylobacter jejuni
  source 1..377: mol_type = genomic DNA
ctgcccggtgctcgaaggttaattgatggggttagcattagcgaagctcttgatcgaagcccgagtaaacggcggccgtaactataacggtcctaaggtagcgaaattccttgtcggttaaataccgacctgcatgaatggcgtaacgagatgggagctgtctcaaagagggatccagtgaaattgtagtggaggtgaaaattcctcctacccgcggcaagacggaaagaccccgtggacctttactacagcttgacactgctacttggataagaatgtgcaggataggtgggaggctttgagtatatgacgccagttgtatatgagccattgttgagataccactctttcttatttgggtagctaaccagcttgag

SEQ ID NO: 24 - DNA, 377 bp, synthetic construct
  source 1..377: mol_type = other DNA
ctgcccggtgctcgaaggttaattgatggggttagcattagcgaagctcttgatcgaagcccgagtaaacggcggccgtaactataacggtcctaaggtagcgaaattccttgtcggttaaataccgacctgcatgaatggcgtaacgagatgggagctgtctcaaagagggatccagtgaaattgtagtggaggtgaaaattcctcctacccgcggcaagacggagagaccccgtggacctttactacagcttgacactgctacttggataagaatgtgcaggataggtgggaggctttgagtatatgacgccagttgtatatgagccattgttgagataccactctttcttatttgggtagctaaccagcttgag